(19) United States
(12) Patent Application Publication
(10) Pub. No.: US 2018/0193169 A1
(43) Pub. Date: Jul. 12, 2018

(54) FEMORAL CUP AND FEMORAL BALL EXTRACTOR

(71) Applicant: HIP INNOVATION TECHNOLOGY, LLC., Boca Raton, FL (US)

(72) Inventors: Zafer TERMANINI, Port Saint Lucie, FL (US); Brian VANHIEL, Smyrna, GA (US)

(21) Appl. No.: 15/742,263

(22) PCT Filed: Jul. 15, 2016
(86) PCT No.: PCT/US16/42443

§ 371 Date: Jan. 5, 2018

Related U.S. Application Data
(60) Provisional application No. 62/197,220, filed on Jul. 27, 2015.

(51) Int. Cl. A61F 2/46

(52) U.S. Cl. CPC A61F 2/4637; A61F 2002/30332; A61F 2002/4641

(57) ABSTRACT

A surgical instrument for separating a femoral cup or a femoral ball from a femoral implant wherein the cup or ball is affixed to the implant by means of a Morse taper. A tapered wedge in the form of a two pronged fork is provided at the distal end of the instrument for placement between the cup or ball and the femoral implant. Then the proximal end of the instrument is struck with a hammer to separate the cup or ball from the femoral implant. The surgical instrument of the invention enables a surgeon to remove the cup or ball without pulling on the femoral implant and, thereby, without disrupting any bone ingrowth in the implanted femoral implant.

### BACKGROUND OF THE INVENTION

### Field of the Invention

The present invention relates to surgical instruments used in connection with a reverse hip prosthesis. More particularly, the invention has to do with a surgical tool for separating a femoral cup from a femoral implant. The invention also can be used in connection with a conventional hip prosthesis to separate a femoral ball from a femoral cup.

### The Related Art

A reverse hip prosthesis is described in U.S. Pat. Nos. 8,313,531 B2 and 8,540,779 B2. The prosthesis and a revision surgery method also are described in U.S. Pat. No. 8,992,627 B2. The disclosures of these three patents are incorporated herein in their entireties by reference.

### SUMMARY OF THE INVENTION

As described in the patents referenced above, the femoral cup is affixed to the femoral implant by means of a Morse taper. The surgical tool of the invention enables a surgeon to remove the femoral cup or femoral ball without pulling on the femoral implant and without disrupting any bone ingrowth in an implanted prosthesis. In the present disclosure, we use the term “femoral cup extractor” from time to time to describe the tool of the invention.

### BRIEF DESCRIPTION OF THE DRAWINGS

FIG. 1 is an elevation view of the surgical tool of the invention.

FIG. 2 is a section view of a femoral cup secured to a femoral implant by means of a Morse taper.

FIG. 3 is a section view of a femoral ball.

FIG. 4 is perspective view of a femoral cup extractor positioned on a femoral cup just prior to extraction of the femoral cup from the femoral implant.

FIG. 5 is a perspective view of a femoral cup extractor positioned relative to a femoral cup and a femoral implant following impacting to loosen the cup from the Morse taper.

### DESCRIPTION OF THE PREFERRED EMBODIMENT

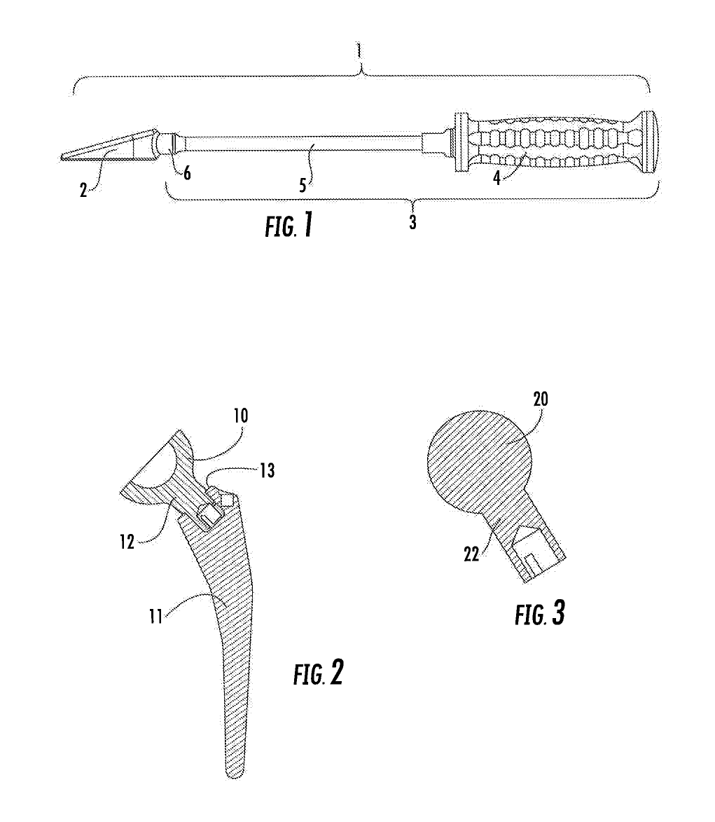

The femoral cup extractor 1 of FIG. 1 has a forked wedge 2 affixed to the distal end of a driver 3. The forked wedge 2 comprises tapered prongs having an upper surface and a lower surface. The prongs are sized to engage on the upper surface an outer convex surface of a femoral cup or femoral ball. On the lower surface the prongs engage the proximal end of the femoral implant. A space between the prongs has sufficient width to receive the neck of the femoral cup or femoral ball. The thickness of the tapered portion of the prongs is thinnest at the distal end of the wedge. The thickness increases in the proximal direction. The driver 3 is comprised of a handle 4 at its proximal end and a shaft 5 with a connector 6 at its distal end. The connector 6 may be threaded, or may be a snap fit or the like as will be understood by those having skill in the art. The proximal end of the shaft 5 is affixed to the distal end of handle 4.

A femoral cup 10 affixed by a Morse taper to a femoral implant 11 is illustrated in section in FIG. 2. The femoral cup 10 has a neck 12 and the proximal end of the femoral implant is designated by the reference numeral 13.

When the femoral implant, with a femoral cup affixed therein, is implanted in the proximal end of a femur, it may be desirable to remove the femoral cup without disturbing the femoral implant. In this way, the femoral implant can be maintained in the femur without disturbing bone ingrowth. Thus, it is desirable to “break” the secure Morse taper connection between the femoral cup and the femoral implant without pulling on the implant. This is achieved by using the surgical tool of the present invention.

According to the method of the invention, the wedge 2 of the femoral cup extractor 1 is positioned relative to the femoral cup 10 and the femoral implant 11 as illustrated in FIG. 4. In this position, the distal end of the wedge is in contact with the proximal end of the femoral implant and the outer convex surface of the femoral cup or femoral ball adjacent to the neck. Thus, the lower surface of the tapered prongs is in contact with the proximal end of the femoral implant and the upper surface of the tapered prongs is in contact with the convex surface of the femoral cup or femoral ball. In this initial position, the neck is partially received within the space between the prongs. Then the proximal end of handle 4 is struck with a hammer to drive wedge 2 toward femoral cup 10 with sufficient force to lift the cup upwardly while keeping the femoral implant securely embedded in the femur. Thus, the cup is no longer affixed to the femoral implant 11 by means of the Morse taper. This position is illustrated in FIG. 5. The cup can then be picked up and removed by hand.

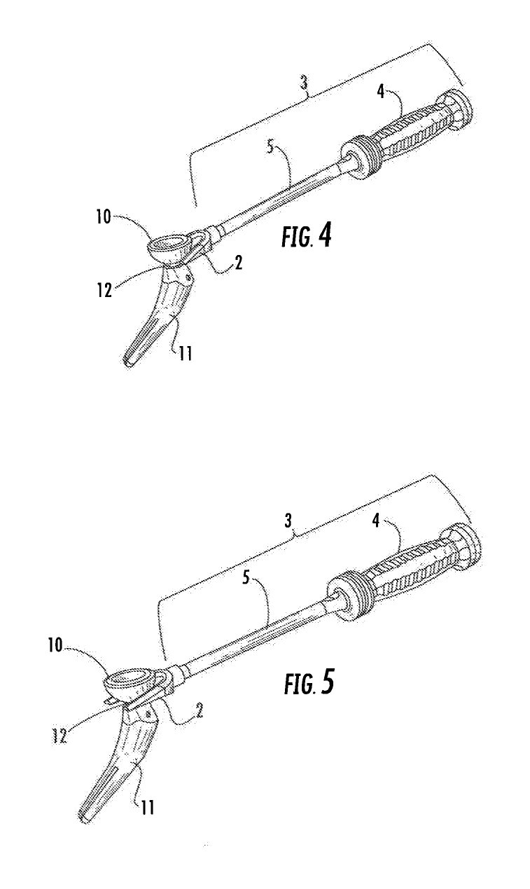

The femoral cup extractor 1 can also be used, following the same method as described above, with a conventional hip implant where a femoral ball is affixed to a femoral implant by means of a Morse taper. A femoral ball 20 having a neck 22 is illustrated in section in FIG. 3.

The femoral cup or ball extractor 1 may be included as a component of a surgical kit which may contain other instruments and/or implant elements.

1. A surgical instrument adapted for removing a femoral cup or a femoral ball from a femoral implant wherein the femoral cup or femoral ball has a neck extending therefrom and the distal end of the neck is affixed by means of a Morse taper to the femoral implant comprising:
a forked wedge having tapered prongs, the prongs having an upper surface and a lower surface and being sized to engage (1) on the upper surface an outer convex surface of the femoral cup or ball adjacent the neck thereof and (2) on the lower surface a proximal end of the femoral implant, the prongs having a space therebetween, the space having a sufficient width to receive the neck, wherein a thickness of the tapered portion of the prongs is thinnest at the distal end of the wedge, the thickness increasing in a proximal direction;
a driver affixed to the proximal end of the wedge, the driver comprising a shaft at its distal end and a handle at its proximal end.

2. A kit containing the surgical instrument of claim 1.

3. A method of using the surgical instrument of claim 1 comprising contacting the distal end of the wedge with the proximal end of the femoral implant and the outer convex surface of the femoral cup or femoral ball adjacent the neck so that the lower surface of the tapered prongs is in contact with the proximal end of the femoral implant and the upper surface of the tapered prongs is in contact with the convex surface of the femoral cup or femoral ball and the neck is partially received within the space between the prongs, tapping the proximal end of the handle with a hammer to cause the femoral cup or ball to be lifted upwardly relative to the femoral implant so that said cup or ball is no longer affixed to the femoral implant by means of the Morse taper.
